(19) United States
(12) Patent Application Publication
(10) Pub. No.: US 2021/0378729 A1
(43) Pub. Date: Dec. 9, 2021

(54) ELECTRODE UNIT AND ENDOSCOPE SYSTEM

(71) Applicant: OLYMPUS CORPORATION, Tokyo (JP)

(72) Inventors: Soichi Ikuma, Tokyo (JP); Yuji Sakai, Tokyo (JP); Tsuyoshi Hayashida, Tokyo (JP); Takayuki Tsukagoshi, Tokyo (JP)

(73) Assignee: OLYMPUS CORPORATION, Tokyo (JP)

(21) Appl. No.: 17/354,386

(22) Filed: Jun. 22, 2021

Related U.S. Application Data
(63) Continuation of application No. PCT/JP2018/048171, filed on Dec. 27, 2018.

(51) Int. Cl. A61B 18/14

(52) U.S. Cl. CPC A61B 18/14; A61B 2018/00982

(57) ABSTRACT

An electrode unit includes: an electrode supporting member provided with a pair of distal end rigid members, surfaces of which are covered by an electrically insulating material, and an elastic region having lower bending rigidity than bending rigidity of each of the pair of distal end rigid members; an electrode configured with electrode bodies respectively protruding downward from the pair of distal end rigid members of the electrode supporting member and a spanning portion that spans between respective lower end portions of the electrode bodies; and an operation member provided in a proximal end rigid member, and configured to move relative to the proximal end rigid member to cause the electrode to move in a direction in which the electrode protrudes relative to the distal end rigid members.

### CROSS REFERENCE TO RELATED APPLICATION

This application is a continuation application of PCT/JP2018/048171 filed on Dec. 27, 2018, the entire contents of which are incorporated herein by this reference.

### BACKGROUND OF THE INVENTION

### 1. Field of the Invention

The present invention relates to an electrode unit and an endoscope system configured to resect or coagulate tissue inside a subject by using a high-frequency current.

### 2. Description of the Related Art

Electrocautery has been known as a technique of resecting or coagulating tissue of a subject such as a human body. For example, Japanese Patent Application Laid-Open Publication No. 2002-95677 discloses a resectoscope system configured to resect or coagulate tissue inside a subject by using a high-frequency current under observation with an endoscope. The technique disclosed in Japanese Patent Application Laid-Open Publication No. 2002-95677 carries out resection or coagulation of tissue by passing a high-frequency current through an electrode formed in a loop shape.

### SUMMARY OF THE INVENTION

An electrode unit according to one aspect of the present invention is configured to resect or coagulate a tissue inside a subject by using a high-frequency current, and includes: an electrode supporting member provided with a pair of distal end rigid members, surfaces of which are covered by an electrically insulating material, and an elastic region having lower bending rigidity than bending rigidity of each of the pair of distal end rigid members, the elastic region being provided on a proximal end side of each of the pair of distal end rigid members; an electrode configured with electrode bodies respectively protruding downward from the pair of distal end rigid members of the electrode supporting member and a spanning portion that spans between respective lower end portions of the electrode bodies; and an operation member provided in a proximal end rigid member, and configured to move relative to the proximal end rigid member to cause the electrode to move in a direction in which the electrode protrudes relative to the pair of distal end rigid members.

An electrode unit according to another aspect of the present invention is configured to resect or coagulate tissue inside a subject by using a high-frequency current under observation with an endoscope, and includes: an electrode supporting member inserted into the subject; a proximal end rigid member joined to a proximal end of the electrode supporting member; a distal end rigid member provided in a distal end portion of the electrode supporting member; an elastic region having lower bending rigidity than bending rigidity of the distal end rigid member and the proximal end rigid member, the elastic region being provided in the electrode supporting member to connect between the distal end rigid member and the proximal end rigid member; an electrode supported by the distal end rigid member and protruding from an external surface of the distal end rigid member; an electrical connection member provided in the proximal end rigid member and electrically connected to the electrode; and an operation member provided in the proximal end rigid member and configured to move relative to the proximal end rigid member to cause the electrode to move relative to the distal end rigid member.

An endoscope system according to yet another aspect of the present invention includes: an electrode unit configured to resect or coagulate tissue inside a subject by using a high-frequency current, the electrode unit including an electrode supporting member provided with a pair of distal end rigid members, surfaces of which are covered by an electrically insulating material, and an elastic region member having lower bending rigidity than bending rigidity of each of the pair of distal end rigid members, the elastic region member being provided on a proximal end side of each of the pair of distal end rigid members, an electrode configured with electrode bodies respectively protruding downward from the pair of distal end rigid members of the electrode supporting member and a spanning portion that spans between respective lower end portions of the electrode bodies; and an operation member provided in a proximal end rigid member, and configured to move relative to the proximal end rigid member to cause the electrode to move in a direction in which the electrode protrudes relative to each of the pair of distal end rigid members; and a telescope for observing an object.

### BRIEF DESCRIPTION OF THE DRAWINGS

FIG. 1 is a diagram showing a schematic configuration of an endoscope system of a first embodiment;

FIG. 2 is a diagram showing an electrode unit of the first embodiment seen along a first axis;

FIG. 3 is a diagram showing the electrode unit of the first embodiment seen along a second axis;

FIG. 4 is a cross-sectional view taken along a line IV-IV in FIG. 3:

FIG. 5 is a cross-sectional view taken along a line V-V in FIG. 4;

FIG. 6 is a diagram explaining an action of a restriction portion of the first embodiment;

FIG. 7 is a diagram explaining an action of the restriction portion of the first embodiment;

FIG. 8 is a diagram showing a manner of resecting tissue by using the electrode unit of the first embodiment;

FIG. 9 is a diagram showing a manner of resecting tissue by using the electrode unit of the first embodiment:

FIG. 10 is a diagram showing a manner of resecting tissue by using the electrode unit of the first embodiment;

FIG. 11 is a diagram showing a configuration of an electrode unit of a second embodiment;

FIG. 12 is a diagram showing a configuration of the electrode unit of the second embodiment; and

FIG. 13 is a diagram showing a configuration of an electrode unit of a third embodiment.

### DETAILED DESCRIPTION OF THE PREFERRED EMBODIMENTS

Hereinafter, preferred embodiments of the present invention are described with reference to the drawings. Note that, in each of the figures used for the following description, components may have different scales so that each of the components has a recognizable size on the figures, and the present invention is not limited to the number, shapes, ratios of size, and relative positional relationships of the components featured in the figures.

### First Embodiment

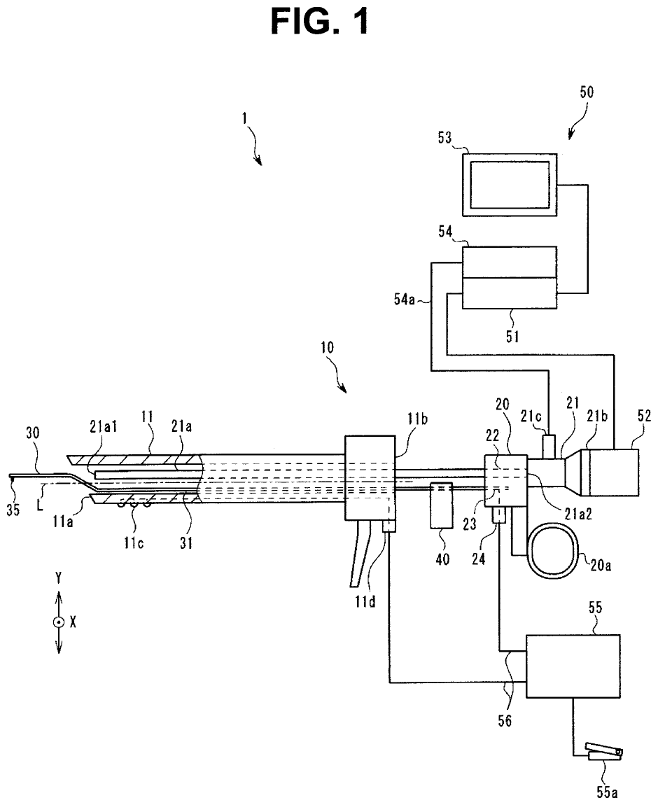

FIG. 1 is a diagram showing a schematic configuration of an endoscope system 1. The endoscope system 1 is a device configured to resect or coagulate tissue inside a subject under observation with an endoscope.

The endoscope system 1 of the present embodiment includes a resectoscope 10, which is an endoscope, an electrode unit 30, and an external device 50. In the present embodiment, the subject is a human body as an example. Although the endoscope in the present embodiment is, as an example, an endoscope typically referred to as a resectoscope, the endoscope may also be a flexible endoscope.

The resectoscope 10 includes a sheath 11, a slider 20, and a telescope 21.

The sheath 11 includes a tubular area along a linear longitudinal axis L. The sheath 11 is an area inserted from the outside of the subject to the inside of the subject during use of the resectoscope 10. The sheath 11 has openings on both ends in a direction along the longitudinal axis L. During use of the resectoscope 10, the telescope 21 and the electrode unit 30 described later are inserted into the sheath 11.

An outer sheath for introducing a perfusate into the subject is arranged on an outer periphery of the sheath 11. A configuration for introducing the perfusate into the subject, such as the outer sheath, is well-known and description of such a configuration is omitted. In the present embodiment, the perfusate is, for example, an electrolyte solution such as physiological saline and is electrically conductive.

Of both ends of the sheath 11 in the direction along the longitudinal axis L, an end on a side to be inserted into the subject is referred to as a “distal end 11a”, and an end on an opposite side to the distal end 11a is referred to as a “proximal end 11b”. The proximal end 11b of the sheath 11 is exposed to the outside of the subject during use of the resectoscope 10.

For the sake of description hereinafter, a first axis X and a second axis Y are defined, which are a pair of axes orthogonal to the longitudinal axis L and orthogonal to each other. In a direction along the first axis X, one side is defined as a right side and the other side is defined as a left side. In a direction along the second axis Y, one side is defined as an upper side and the other side is defined as a lower side. In the present embodiment, in an image picked up by using the telescope 21, a horizontal direction is substantially parallel to the first axis X, and a perpendicular direction is substantially parallel to the second axis Y, as an example. The upper side and the right side are an upper side and a right side in the image picked up by using the telescope 21.

A collection electrode 11c configured with an electrically conductive material is exposed at least to the surface in vicinity of the distal end 11a of the sheath 11. Note that it may also be configured that the entire sheath 11 is configured with an electrically conductive material such as a metal and the entire surface of the sheath 11 functions as the collection electrode 11c.

A sheath connector 11d is provided in vicinity of the proximal end 11b of the sheath 11. The sheath connector 11d is electrically connected to the collection electrode 11c. A cable 56 is connected to the sheath connector 11d. The cable 56 electrically connects between the sheath connector 11d and a high-frequency power control device 55 of the external device 50.

The slider 20 is arranged on a side of the proximal end 11b of the sheath 11. The slider 20 moves relative to the sheath 11 in the direction along the longitudinal axis L. The slider 20 is provided with a handle 20a. When a user applies a force to the handle 20a with fingers, the slider 20 moves relative to the sheath 11 in the direction along the longitudinal axis L. Note that a mechanism for guiding the slider 20 in a relatively movable manner to the sheath 11 is similar to the mechanism of the conventional resectoscope 10, and therefore illustration and description of the mechanism are omitted.

The slider 20 includes a scope holding portion 22, an electrode unit holding portion 23, and an electrode connector 24. The scope holding portion 22 holds the telescope 21.

The telescope 21 is an area for optically observing the inside of the subject. The telescope 21 is provided with an elongated insertion portion 21a, an eye piece 21b, and alight source connection portion 21c. The insertion portion 21a is inserted into the sheath 11 in a state in which the telescope 21 is fixed onto the scope holding portion 22.

An observation window and an illumination light emission window are provided on a distal end portion 21al of the insertion portion 21a. The eye piece 21b and the light source connection portion 21c are provided on a proximal end portion 21a2 of the insertion portion 21a.

An image pickup unit 52 is attached to the eye piece 21b. The image pickup unit 52 is electrically connected to a video processor 51 of the external device 50. An image display device 53 is electrically connected to the video processor 51. One end of an optical fiber cable 54a is connected to the light source connection portion 21c. The other end of the optical fiber cable 54a is connected to a light source device 54 of the external device 50.

A field of view from the observation window provided on the distal end portion 21a1 of the insertion portion 21a is picked up by the image pickup unit 52 and displayed on the image display device 53. Illumination light emitted from the light source device 54 is emitted from the illumination light emission window provided on the distal end portion 21a1 of the insertion portion 21a. Configurations of the telescope 21 and the external device 50 connected to the telescope 21 are similar to the configurations in the conventional resectoscope 10, and therefore detailed description of the configurations is omitted.

The electrode unit holding portion 23 holds the electrode unit 30 described later. The electrode connector 24 is electrically connected to the electrode unit 30 held by the electrode unit holding portion 23. A cable 56 is connected to the electrode connector 24. The cable 56 electrically connects between the electrode connector 24 and the high-frequency power control device 55 of the external device 50.

The electrode unit 30 includes an area inserted into the sheath 11 in a state of being fixed onto the electrode unit holding portion 23. The slider 20 moves together with the telescope 21 and the electrode unit 30, relative to the sheath 11 in the direction along the longitudinal axis L. A part of the electrode unit 30 may protrude from the distal end 11a of the sheath 11. As described later, an electrode 35 is provided in the area of the electrode unit 30 protruding from the distal end 11a of the sheath 11.

The electrode unit 30, the collection electrode 11c, and the high-frequency power control device 55 configure a so-called bipolar electro-surgical apparatus. The high-frequency power control device 55 is provided with a switch 55a. The switch 55a is, for example, a foot switch operated by a user's foot. The high-frequency power control device 55 is configured to switch between presence and absence of output of a high-frequency current according to an operation on the switch 55a.

The high-frequency current outputted from the high-frequency power control device 55 flows among the electrode 35, the perfusate, and the collection electrode 11c inside the subject. In a state in which the high-frequency power control device 55 outputs the high-frequency current, tissue in the subject in contact with the electrode 35 generates heat, whereby the tissue is resected or coagulated.

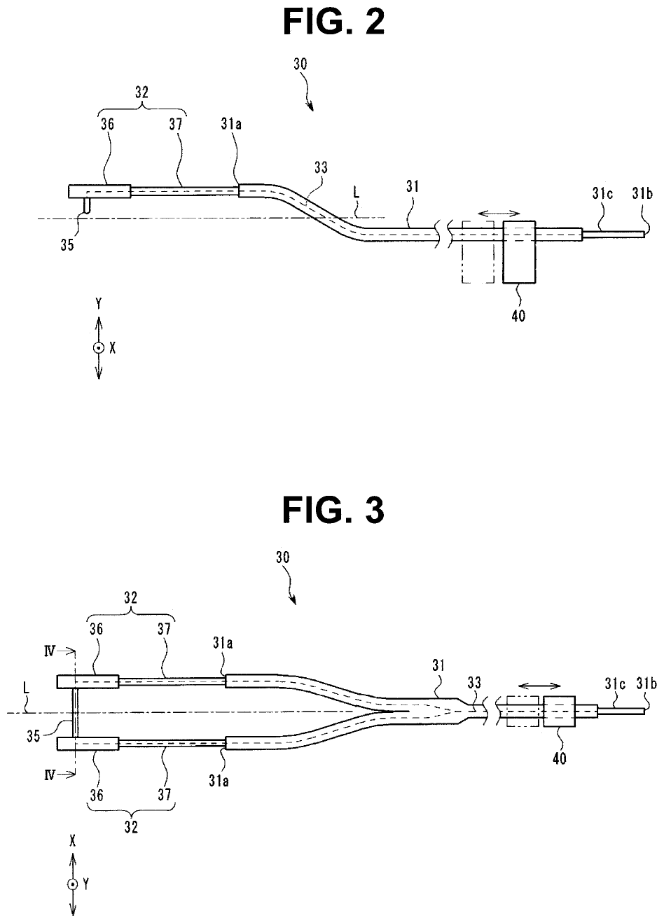

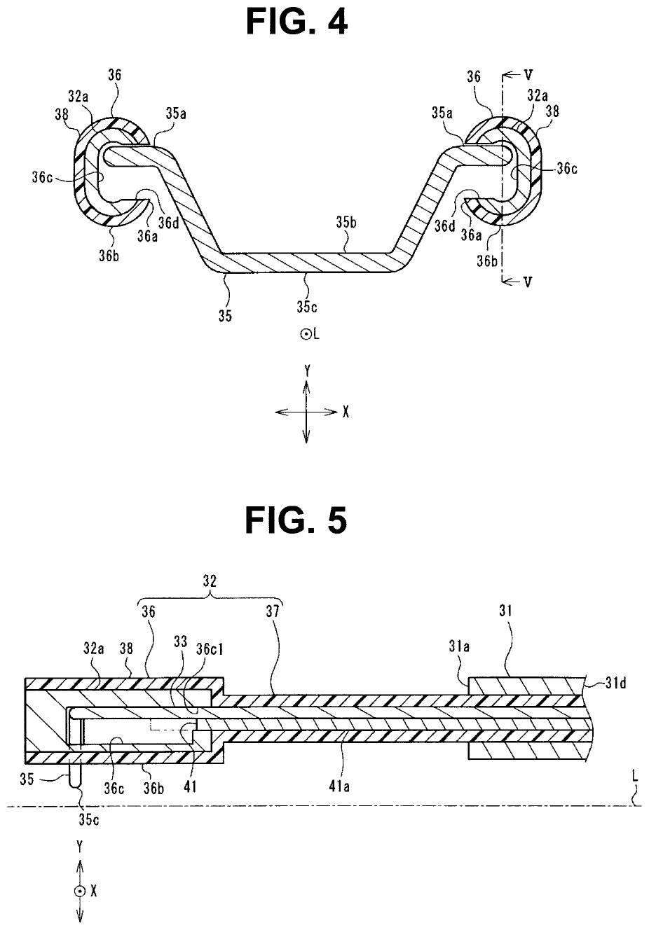

FIG. 2 is a diagram showing the electrode unit 30 seen from left along the first axis X. In FIG. 2, an upper side of the diagram is the upper side. FIG. 3 is a diagram showing the electrode unit 30 seen from below along the second axis Y. In FIG. 3, an upper side of the diagram is the left side. FIG. 4 is a cross-sectional view taken along a line IV-IV in FIG. 3. In FIG. 4, an upper side of the diagram is the upper side, and a right side of the diagram is the left side. FIG. 5 is a cross-sectional view taken along a line V-V in FIG. 4. In FIG. 5, an upper side of the diagram is the upper side.

As shown in FIG. 2 and FIG. 3, the electrode unit 30 has an elongated shape, the longitudinal direction of which is a direction along the longitudinal axis L. The electrode unit 30 includes a proximal end rigid portion 31, an electrode supporting portion 32, the electrode 35 and an operation portion 40.

The proximal end rigid portion 31 is an area fixed to the electrode unit holding portion 23 of the resectoscope 10. The electrode supporting portion 32 described later is joined to a distal end 31a of the proximal end rigid portion 31. An electrical connection portion 31c is provided on a proximal end 31b of the proximal end rigid portion 31. The electrical connection portion 31c is electrically connected to the electrode connector 24 of the resectoscope 10 in a state in which the proximal end rigid portion 31 is fixed to the electrode unit holding portion 23. The electrical connection portion 31c is electrically connected to the electrode 35 via an electrically conductive wire 33 inserted into the electrode unit 30.

The electrode supporting portion 32 supports the electrode 35. The electrode supporting portion 32 is an area protruding from the distal end 11a of the sheath 11 during use of the resectoscope 10. The electrode supporting portion 32 includes one or two distal end rigid portions 36 and one or two elastic regions 37.

The elastic region 37 joins a proximal end of the distal end rigid portion 36 with a distal end of the proximal end rigid portion 31. The bending rigidity of the elastic region 37 is lower than the bending rigidity of the distal end rigid portion 36 and the proximal end rigid portion 31.

The electrode 35 includes an electrically conductive linear member such as a metal wire. The electrode 35 protrudes from a surface of the distal end rigid portion 36.

The electrode 35 has a loop shape protruding from one point on the surface of the distal end rigid portion 36 to the outside of the distal end rigid portion 36, and entering from another point to the inside of the distal end rigid portion 36. More specifically, the electrode 35 includes, in two points spaced apart from each other on the surface of the distal end rigid portion 36, a pair of base portions 35a supported by the distal end rigid portion 36 and a spanning portion 35b connecting between the pair of base portions 35a in a state of being spaced apart from the surface of the distal end rigid portion 36.

As shown in FIG. 4, the spanning portion 35b is substantially angled-U shaped or substantially U shaped when seen in a direction along the longitudinal axis L. When seen in the direction along the first axis X, an apex portion 35c of the spanning portion 35b protrudes from the base portion 35a in a direction intersecting the longitudinal axis L.

The pair of base portions 35a is electrically connected to the wire 33 inside the distal end rigid portion 36. As shown in FIG. 4 and FIG. 5, in the present embodiment, the wire 33 and the electrode 35 are configured with the same metallic linear member, as an example.

The operation portion 40 is provided in the proximal end rigid portion 31. The operation portion 40 is positioned more on the proximal end side than the proximal end 11b of the sheath 11 in a state in which the electrode unit 30 is inserted into the sheath 11. In other words, in the state in which the distal end 11a of the sheath 11 and the electrode supporting portion 32 of the electrode unit 30 are inserted into the subject, the operation portion 40 is positioned outside of the subject.

The operation portion 40 is movable relative to the proximal end rigid portion 31. By moving the operation portion 40 relative to the proximal end rigid portion 31, the electrode 35 is caused to move relative to the distal end rigid portion 36.

Detailed configurations of the electrode supporting portion 32, the electrode 35, and the operation portion 40 are described. The electrode supporting portion 32 of the present embodiment is provided with two distal end rigid portions 36. Each of the distal end rigid portions 36 has a columnar external shape, the longitudinal direction of which is a direction along the longitudinal direction L.

The two distal end rigid portions 36 are arranged in substantially the same position in the direction along the longitudinal direction L, and arranged to be spaced apart from each other in the direction along the first axis X (lateral direction). In other words, the two distal end rigid portions 36 are arranged such that an overlapping part exists when seen in the direction along the first axis X. Therefore, the two distal end rigid portions 36 respectively have opposed faces 36a being opposed to each other in the direction along the first axis X.

Note that the expression “faces being opposed to each other” as used herein refers to a surface oriented substantially to the left side of the distal end rigid portion 36 arranged on the right side, and a surface oriented substantially to the right side of the distal end rigid portion 36 arranged on the left side. In other words, the opposed faces 36a are areas facing a space between the two distal end rigid portions 36. Therefore, the opposed faces 36a of the two distal end rigid portions 36 are not required to have respective areas parallel to each other.

Inside each of the distal end rigid portions 36, a hollow portion 36c is formed. As shown in FIG. 5, an opening portion 36cl is opened on an inner wall surface on the proximal end side of the hollow portion 36c. A wire 33 and a restriction portion 41 described later are inserted into the opening portion 36c1.

In each of the distal end rigid portions 36, a through hole 36d penetrating from the opposed face 36a to the hollow portion 36c is formed. The wire 33 protrudes through the through hole 36d to the outside of the distal end rigid portion 36. An area of the wire 33 inserted into the through hole 36d acts as the base portion 35a of the electrode 35. The through hole 36d is an elongated hole, the longitudinal direction of which corresponds to a direction along the second axis Y (vertical direction).

More precisely, each of the distal end rigid portions 36 is configured with a ceramic pipe 32a and a covering portion 38. The ceramic pipe 36c and the covering portion 38 are electrically insulating. Inside the ceramic pipe 32a, a hollow portion 36c is formed. The covering portion 38 is a tube made of resin and covers the ceramic pipe 32a. The through hole 36d penetrates the ceramic pipe 32a and the covering portion 38.

The electrode supporting portion 32 of the present embodiment is provided with two elastic regions 37, as an example. The two elastic regions 37 are connected respectively to the proximal ends of the two distal end rigid portions 36. Note that the electrode supporting portion 32 may also be configured to have one elastic region 37 connected to the proximal ends of both of the two distal end rigid portions 36.

The elastic region 37 of the present embodiment is configured with the covering portion 38, which is a tube made of resin. In the present embodiment, the covering portion 38 of the distal end rigid portion 36 and the covering portion 38 of the elastic region 37 are the same member continuous in the direction along the longitudinal axis L, as an example. The wire 33 is inserted into the covering portion 38 of the elastic region 37. In other words, in the present embodiment, the ceramic pipe 32a inserted into the covering portion 38 plays a role in making the bending rigidity of the distal end rigid portions 36 higher than the bending rigidity of the elastic region 37.

The proximal end rigid portion 31 of the present embodiment is configured with the covering portion 38, which is a tube made of resin, and a metal pipe 31d. In the present embodiment, the covering portion 38 of the proximal end rigid portion 31 and the covering portion 38 of the elastic region 37 are the same member continuous in the direction along the longitudinal axis L, as an example. The wire 33 is inserted into the covering portion 38 of the proximal end rigid portion 31. The metal pipe 31d covers the outer periphery of the covering portion 38. In other words, in the present embodiment, the metal pipe 31d plays a role in making the bending rigidity of the proximal end rigid portions 31 higher than the bending rigidity of the elastic region 37.

The pair of base portions 35a of the electrode 35 are arranged respectively on the two distal end rigid portions 36. In other words, the electrode 35 is the metal wire 33 spanning between the two distal end rigid portions 36.

The pair of base portions 35a are arranged to protrude along the first axis X, respectively from the through holes 36d provided on the opposed faces 36a of the two distal end rigid portions 36. The pair of base portions 35a are arranged in substantially the same position in the direction along the longitudinal axis L. In other words, the pair of base portions 35a protrude respectively from the pair of opposed faces 36 along the first axis X, toward each other.

The spanning portion 35b connects between distal end portions of the pair of base portions 35a. The spanning portion 35b is curved in a downward convex shape from the pair of base portions 35a, when seen in the direction along the longitudinal axis L. As shown in FIG. 4 and FIG. 5, the apex portion 35c of the spanning portion 35b is positioned on the lower side of a lower end face 36b, facing the lower side, of the two distal end rigid portions 36.

The electrode 35 having the configuration described in the foregoing is exposed to the outside only within a space between the two distal end rigid portions 36 when seen in the direction along the second axis Y. In other words, an area of the electrode 35 exposed to the outside is arranged not to overlap the two distal end rigid portions 36 when seen in the direction along the second axis Y.

Since the through hole 36d into which the base portion 35a is inserted is an elongated hole, the longitudinal direction of which is along the second axis Y as described above, the electrode 35 is movable relative to the distal end rigid portion 36 in a direction along the second axis Y. When the electrode 35 moves relative to the distal end rigid portion 36 in the direction along the second axis Y, a downward protrusion amount of the apex 35c of the electrode 35 from the lower end face 36b is changed.

The electrode unit 30 of the present embodiment is provided with the restriction portion 41 arranged inside the hollow portion 36c of each of the distal end rigid portions 36. The restriction portion 41 protrudes from the opening portion 36c1 into the hollow portion 36c, and is movable relative to the distal end rigid portion 36. The restriction portion 41 advances and retreats relative to the distal end rigid portion 36 in the direction along the longitudinal axis L.

The restriction portion 41 includes a joint portion 41a that extends toward the proximal end side and is inserted into the elastic region 37 and the proximal end rigid portion 31. The joint portion 41a is joined to the operation portion 40. The restriction portion 41 moves inside the hollow portion 36c in association with movement of the operation portion 40 relative to the proximal end rigid portion 31.

The restriction portion 41 moves inside the hollow portion 36c relative to the distal end rigid portion 36, to change a movable range of the electrode 35 in the protrusion direction (vertical direction). The restriction portion 41 of the present embodiment advances and retreats inside the hollow portion 36c in the direction along the longitudinal axis L, in a state of being in contact with a lower face of the wire 33. A greater protrusion amount of the restriction portion 41 from the opening portion 36c1 into the hollow portion 36c leads to a narrower movable range of the wire 33 in the vertical direction, whereby the downward protrusion amount of the electrode 35 from the lower end face 36b is changed.

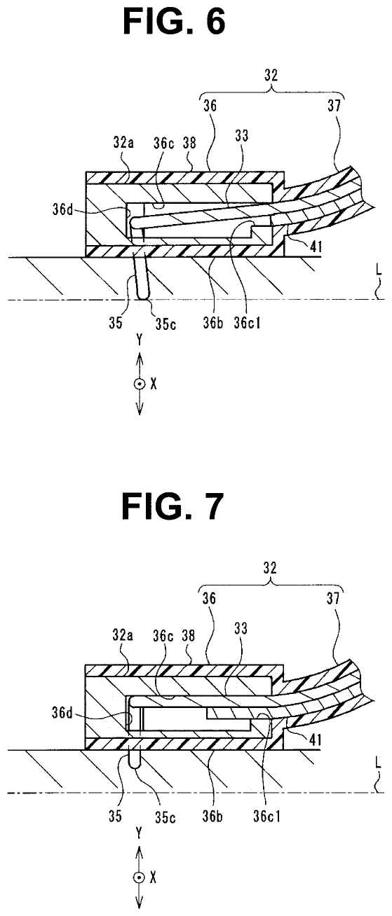

FIG. 6 shows a state in which the restriction portion 41 is positioned on the proximalmost side with respect to the distal end rigid portion 36. FIG. 7 shows a state in which the restriction portion 41 is positioned on the distalmost side with respect to the distal end rigid portion 36. In the electrode unit 30 of the present embodiment, by moving the operation portion 40 to the proximal end side relative to the proximal end rigid portion 31, the restriction portion 41 moves to the proximal end side relative to the distal end rigid portion 36. In the electrode unit 30 of the present embodiment, by moving the operation portion 40 to the distal end side relative to the proximal end rigid portion 31, the restriction portion 41 moves to the distal end side with respect to the distal end rigid portion 36.

FIG. 6 and FIG. 7 show a state in which the lower end face 36b of the distal end rigid portion 36 is brought into contact with the tissue. When the lower end face 36b of the distal end rigid portion 36 is brought into contact with the tissue, the elastic region 37 connected to the proximal end of the distal end rigid portion 36 is curved downward in a convex shape. Due to the curvature of the elastic region 37, the wire 33 protruding from the elastic region 37 into the hollow portion 36c is applied with a force moving the distal end downward, with the opening portion 36cl as a fulcrum. The electrode 35 is connected to the distal end of the wire 33 in the hollow portion 36c. Therefore, as shown in FIG. 6 and FIG. 7, when the lower end face 36b of the distal end rigid portion 36 is brought into contact with the tissue, the electrode 35 is positioned at the lower end of the movable range in the vertical direction with respect to the distal end rigid portion 36.

When the restriction portion 41, which is in contact with a lower side of the wire 33, is positioned on the proximalmost side as shown in FIG. 6, the movable range of the distal end of the wire 33 in the vertical direction within the hollow portion 36c is the broadest. On the other hand, when the restriction portion 41 moves to the distal end side as shown in FIG. 7, the restriction portion 41 advances to the lower side of the wire 33 within the hollow portion 36c, whereby the movable range of the distal end of the wire 33 in the vertical direction becomes narrower. More specifically, when the restriction portion 41 moves to the distal end side, the lower end of the movable range of the distal end of the wire 33 within the hollow portion 36c moves to the upper side. The expression “movable range of the distal end of the wire 33 within the hollow portion 36c” refers to the movable range in the vertical direction of the electrode 35 with respect to the distal end rigid portion 36.

Therefore, in the present embodiment, when the restriction portion 41 is positioned on the proximalmost side as shown in FIG. 6, the downward protrusion amount of the apex portion 35c of the electrode 35 from the lower end face 36b is the greatest. In addition, in the present embodiment, as the restriction portion 41 moves toward the distal end side as shown in FIG. 7, the downward protrusion amount of the apex portion 35c of the electrode 35 from the lower end face 36b becomes smaller.

The operation portion 40 is provided in the proximal end rigid portion 31. The operation portion 40 is positioned more on the proximal end side than the proximal end 11b of the sheath 11 in a state in which the electrode unit 30 is inserted into the sheath 11. In other words, in the state in which the distal end 11a of the sheath 11 and the electrode supporting portion 32 of the electrode unit 30 are inserted into the subject, the operation portion 40 is positioned outside of the subject.

The operation portion 40 is movable relative to the proximal end rigid portion 31. By moving the operation portion 40 relative to the proximal end rigid portion 31, the electrode 35 is caused to move relative to the distal end rigid portion 36, whereby the downward protrusion amount of the apex portion 35c of the electrode 35 from the lower end face 36b is changed.

As described in the foregoing, in the electrode unit 30 of the present embodiment, the protrusion amount of the apex portion 35c of the electrode 35 from the lower end face 36b of the distal end rigid portion 36 can be changed by moving the operation portion 40.

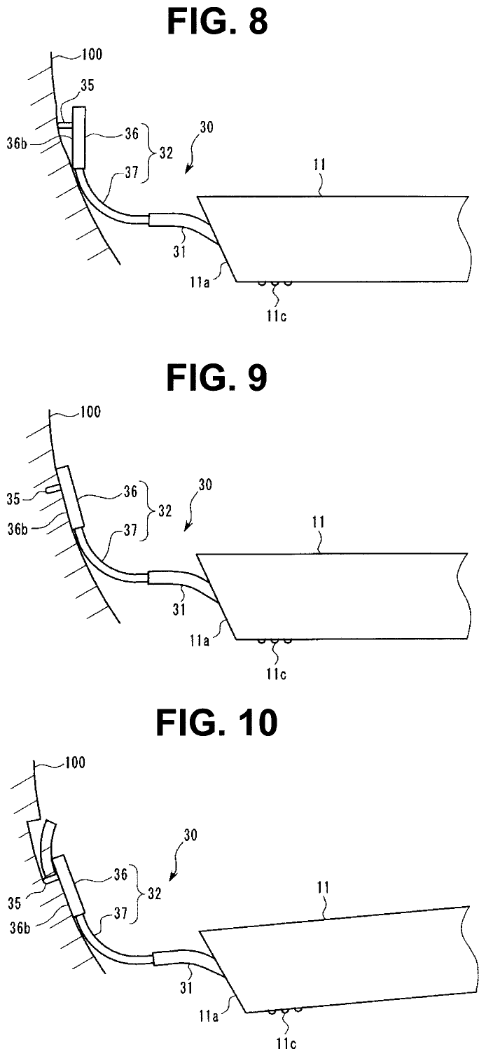

FIG. 8, FIG. 9, and FIG. 10 show a schematic view of a manner of resecting tissue in an organ 100 in the subject by using the electrode unit 30 and the endoscope system 1 of the present embodiment.

In the case of resecting tissue inside the organ 100 by using the electrode unit 30, a user first orients the electrode supporting portion 32 such that the lower end face 36b of the distal end rigid portion 36 faces the tissue inside the organ 100. And then the user brings the electrode supporting portion 32 into contact with a wall surface of the organ 100 such that the electrode 35 protruding from the lower end face 36b of the distal end rigid portion 36 is in contact with the tissue, as shown in FIG. 8. Note that the method of inserting the electrode unit 100 and the sheath 11 of the resectoscope 10 into the organ 100 as well as the method of filling the organ 100 with the perfusate are the same as the method in the case of the conventional electrode unit, and therefore description of the methods is omitted.

Next, the user operates the switch 55a to start output of the high-frequency current from the high-frequency power control device 55. As a result, the high-frequency current flows from the electrode 35 to the collection electrode 11c through the perfusate, whereby the tissue in contact with the electrode 35 generates heat and is cut off. When the tissue is cut off due to the start of output of the high-frequency current, the electrode 35 enters the tissue as shown in FIG. 9.

As described above, the electrode 35 is arranged not to overlap the distal end rigid portions 36 when seen in the direction along the second axis Y (from the lower side). Therefore, when the electrode 35 enters the tissue to a predetermined depth, the distal end rigid portion 36 comes into contact with tissue not being cut off by the electrode 35. Therefore, in the present embodiment, even when the force with which the user presses the electrode supporting portion 32 against the wall surface of the organ 100 varies, the electrode 35 can be prevented from further entering the tissue, from the state in which the distal end rigid portions 36 is in contact with the tissue.

As shown in FIG. 10, the user moves the resectoscope 10 to move the electrode supporting portion 32 along the wall surface of the organ 100. As a result, the electrode 35 moves in a direction along the wall surface inside the tissue, whereby a tissue piece of a predetermined thickness is resected.

As described above, even when the force with which the user presses the electrode supporting portion 32 against the wall surface of the organ 100 varies, the depth to which the electrode 35 enters the tissue is kept constant. In the present embodiment, even when movement of the resectoscope 10 by the user does not follow the shape of the wall surface of the organ 100 and a distance between the wall surface of the organ 100 and the distal end 11a of the sheath 11 varies, the elastic region 37 elastically deforms to keep the state in which the distal end rigid portions 36 is in contact with the tissue. As described above, when the distal end rigid portions 36 is in contact with the tissue, the depth to which the electrode 35 enters the tissue is kept constant.

As explained in the foregoing, the electrode unit 30 and the endoscope system 1 of the present embodiment can keep constant the depth to which the electrode 35 enters the tissue, even when a trajectory of the electrode 35 moved by the user staggers, or when the force applied by the user to the electrode 35 varies.

In the electrode unit 30 and the endoscope system 1 of the present embodiment, the user can change the protrusion amount of the apex portion 35c of the electrode 35 from the lower end face 36b of the distal end rigid portion 36 by operating the operation portion 40 positioned outside of the subject. In other words, in the electrode unit 30 and the endoscope system 1 of the present embodiment, the user can easily change the depth to which the electrode 35 enters the tissue, by operating the operation portion 40.

Therefore, the electrode unit 30 and the endoscope system 1 of the present embodiment facilitate control of the thickness of the resected tissue.

### Second Embodiment

A second embodiment of the present invention is described hereinafter. In the following description, only differences from the first embodiment are explained, and components similar to the components of the first embodiment are denoted by the same reference symbols and description of such components is omitted as appropriate.

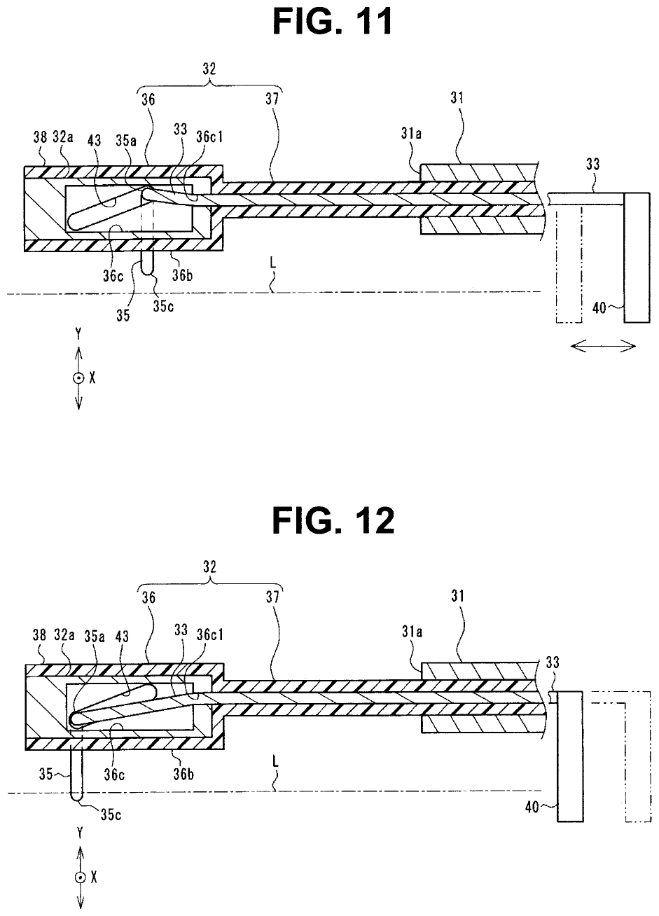

The second embodiment is different from the first embodiment in a configuration of the electrode unit 30. The electrode unit 30 of the second embodiment shown in FIG. 11 and FIG. 12 includes a proximal end rigid portion 31, an electrode supporting portion 32, an electrode 35, an operation portion 40, and a redirection portion 43.

In a similar manner to the first embodiment, the proximal end rigid portion 31 is an area fixed to the electrode unit holding portion 23 of the resectoscope 10. The electrode supporting portion 32 is joined to a distal end 31a of the proximal end rigid portion 31. An electrical connection portion 31c is provided at a proximal end 31b of the proximal end rigid portion 31.

The electrode supporting portion 32 includes one or two distal end rigid portions 36 and one or two elastic regions 37 that are connected in a direction along the longitudinal axis L. In the present embodiment, in a similar manner to the first embodiment, the electrode supporting portion 32 is provided with two distal end rigid portions 36 and two elastic regions 37 as an example. In other words, the two distal end rigid portions 36 respectively have opposed faces 36a being opposed to each other in the direction along the first axis X. The wire 33 is inserted into the proximal end rigid portion 31, the elastic region 37, and the distal end rigid portion 36.

The electrode 35 protrudes from the distal end rigid portion 36. The electrode 35 is connected to the wire 33 inside the distal end rigid portion 36. In the present embodiment, the wire 33 and the electrode 35 are configured with the same metallic linear member, as an example. The apex portion 35c of the electrode 35 protrudes downward from the lower end face 36b of the distal end rigid portion 36, along the second axis Y.

Inside each of the distal end rigid portions 36, a hollow portion 36c is formed. An opening portion 36c1 is opened on an inner wall surface on the proximal end side of the hollow portion 36c. The wire 33 is inserted into the opening portion 36cl.

Inside each of the distal end rigid portions 36, the redirection portion 43 is formed in a shape of an elongated hole. The redirection portion 43 is a through hole penetrating in the direction along the first axis X so as to communicate from the opposed face 36a with the hollow portion 36c.

The longitudinal direction of the redirection portion 43, which is an elongated hole, is inclined with respect to the longitudinal axis L when seen in the direction along the first axis X. In the present embodiment, the redirection portion 43, which is an elongated hole, is inclined upward with respect to the longitudinal axis L as the redirection portion 43 approaches the proximal end side, when seen in the direction along the first axis X. In other words, when seen in the direction along the first axis X, the longitudinal direction of the redirection portion 43, which is an elongated hole, intersects both the protrusion direction of the electrode 35 (downward direction) and the longitudinal axis L.

The pair of base portions 35a of the electrode 35 are inserted into the redirection portion 43 opened on each of the opposed faces 36a of the two distal end rigid portions 36.

The operation portion 40 is movable relative to the proximal end rigid portion 31. The operation portion 40 of the present embodiment transmits to the electrode 35 a force causing to move in the direction along the longitudinal axis L with respect to the distal end rigid portion 36.

More specifically, the operation portion 40 of the present embodiment is joined to the wire 33. The wire 33 moves within the electrode unit 30 in the direction along the longitudinal axis L in association with movement of the operation portion 40 relative to the proximal end rigid portion 31. Since the electrode 35 is connected to the distal end of the wire 33 as described above, the electrode 35 moves in the direction along the longitudinal axis L with respect to the distal end rigid portion 36 in association with movement of the operation portion 40 relative to the proximal end rigid portion 31.

The base portion 35a of the electrode 35 is inserted into the redirection portion 43, which is an elongated hole, the longitudinal direction of which is inclined with respect to the longitudinal axis L. Therefore, when a force is applied from the operation portion 40 to the electrode 35, the redirection portion 43 acts as a cam hole configured to redirect the force transmitted from the operation portion 40 to the electrode 35.

The redirection portion 43 of the present embodiment redirects the force along the longitudinal axis L applied from the operation portion 40 to the electrode 35, to a direction intersecting both the protrusion direction of the electrode 35 (downward direction) and the longitudinal axis L.

More specifically, as the operation portion 40 moves toward the proximal end side, the electrode 35 moves toward the proximal end side and the upper side along the redirection portion 43, when seen in the direction along the first axis X. In this case, as the operation portion 40 moves toward the proximal end side, the downward protrusion amount of the apex portion 35c of the electrode 35 from the lower end face 36b becomes smaller. FIG. 11 shows a state in which the operation portion 40 is positioned on the proximalmost side of the movable range.

As the operation portion 40 moves toward the distal end side, the electrode 35 moves toward the distal end side and the lower side along the redirection portion 43, when seen in the direction along the first axis X. In this case, as the operation portion 40 moves toward the distal end side, the downward protrusion amount of the apex portion 35c of the electrode 35 from the lower end face 36b becomes greater. FIG. 12 shows a state in which the operation portion 40 is positioned on the proximalmost side of the movable range.

As described in the foregoing, the electrode unit 30 of the present embodiment is provided with: the operation portion 40 configured to push and pull the electrode 35 along the longitudinal axis L; and the redirection portion 43 which is a cam configured to redirect the moving direction of the electrode 35 to the direction inclined with respect to the longitudinal axis L when seen in the direction along the first axis X.

In the electrode unit 30 of the present embodiment, the protrusion amount of the apex portion 35c of the electrode 35 from the lower end face 36b of the distal end rigid portion 36 can be changed by moving the operation portion 40. In other words, in the electrode unit 30 and the endoscope system 1 of the present embodiment, the user can easily change the depth to which the electrode 35 enters the tissue, by operating the operation portion 40.

In a similar manner to the first embodiment, the electrode unit 30 and the endoscope system 1 of the present embodiment, in which the electrode supporting portion 32 includes the distal end rigid portion 36 and the elastic region 37, can keep constant the depth to which the electrode 35 enters the tissue, even when a trajectory of the electrode 35 moved by the user staggers, or when the force applied by the user to the electrode 35 varies.

### Third Embodiment

A third embodiment of the present invention is described hereinafter. In the following description, only differences from the second embodiment are explained, and components similar to the components of the second embodiment are denoted by the same reference symbols and description of such components is omitted as appropriate.

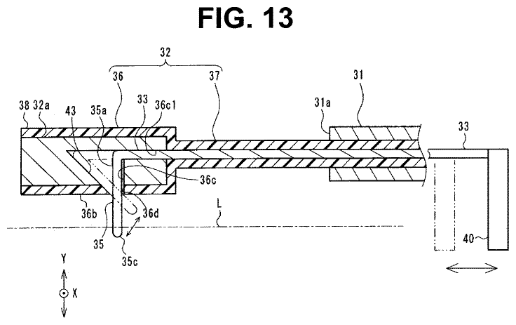

The electrode unit 30 of the third embodiment shown in FIG. 13 is different from the second embodiment in configurations of the distal end rigid portion 36, the electrode 35, and the redirection portion 43.

In the distal end rigid portion 36, a through hole 36d penetrating from the lower end face 36b to the hollow portion 36c is formed. The base portion 35a of the electrode 35 is inserted into the through hole 36d. In other words, in the present embodiment, the base portion 35a of the electrode 35 protrudes downward from the lower end face 36b of the distal end rigid portion 36.

The redirection portion 43 is a slope formed inside the hollow portion 35. The redirection portion 43, which is a slope, is formed on an inner wall surface on the distal end side of the hollow portion 36c. The redirection portion 43 is inclined downward with respect to the second axis Y and the longitudinal axis L as the redirection portion 43 approaches the proximal end side, when seen in the direction along the first axis X. The redirection portion 43 is provided in a position in the hollow portion 35 with which the base portion 35a of the electrode 35 comes into contact when the electrode 35 moves toward the distal end side.

When the operation portion 40 is positioned at the proximalmost end of the movable range, the base portion 35a of the electrode 35 is spaced apart from the redirection portion 43 as shown by a solid line in FIG. 13. In the state in which the base portion 35a is spaced apart from the redirection portion 43, the longitudinal direction of the base portion 35a is substantially parallel to the second axis Y, when seen in the direction along the first axis X. In other words, the longitudinal direction of the base portion 35a is substantially orthogonal to the longitudinal axis L. In other words, in the state in which the operation portion 40 is positioned at the proximalmost end of the movable range, the apex portion 35c of the electrode 35 protrudes in a direction substantially orthogonal to the lower end face 36b of the distal end rigid portion 36. The shortest distance between the lower end face 36b and the apex portion 35c of the electrode 35 becomes the greatest in this case.

When the operation portion 40 is positioned at the distalmost end of the movable range, the base portion 35a of the electrode 35 is oriented such that the longitudinal direction is inclined with respect to the second axis Y and the longitudinal axis L along the redirection portion 43 as shown by a dashed-two dotted line in FIG. 13. In other words, the redirection portion 43, which is a slope, redirects the force transmitted from the operation portion 40 to the electrode 35, to move the base portion 35a substantially orthogonal to the longitudinal axis L in a direction of falling in the direction along the longitudinal axis L.

In the state in which the operation portion 40 is positioned at the distalmost end of the movable range, the apex portion 35c of the electrode 35 protrudes from the lower end face 36b of the distal end rigid portion 36 in a direction inclined with respect to the second axis Y. The shortest distance between the lower end face 36b and the apex portion 35c of the electrode 35 becomes the smallest in this case.

As described in the foregoing, the electrode unit 30 of the present embodiment is provided with: the operation portion 40 configured to push and pull the electrode 35 along the longitudinal axis L; and the redirection portion 43 configured to redirect the moving direction of the apex portion 35c of the electrode 35 to the direction intersecting the longitudinal axis L when seen in the direction along the first axis X.

In the electrode unit 30 of the present embodiment, the protrusion amount of the apex portion 35c of the electrode 35 from the lower end face 36b of the distal end rigid portion 36 can be changed by moving the operation portion 40. In other words, in the electrode unit 30 and the endoscope system 1 of the present embodiment, the user can easily change the depth to which the electrode 35 enters the tissue, by operating the operation portion 40.

In a similar manner to the first embodiment, the electrode unit 30 and the endoscope system 1 of the present embodiment, in which the electrode supporting portion 32 includes the distal end rigid portion 36 and the elastic region 37, can keep constant the depth to which the electrode 35 enters the tissue, even when a trajectory of the electrode 35 moved by the user staggers, or when the force applied by the user to the electrode 35 varies.

The present invention is not limited to the aforementioned embodiments and can be modified as appropriate without departing from the gist or spirit of the present invention that can be read from the claims and the specification as a whole. An electrode unit and an endoscope system with such a modification are also encompassed in the technical scope of the present invention.

What is claimed is:

1. An electrode unit configured to resect or coagulate tissue inside a subject by using a high-frequency current, comprising:
an electrode supporting member provided with a pair of distal end rigid members, surfaces of which are covered by an electrically insulating material, and an elastic region having lower bending rigidity than bending rigidity of each of the pair of distal end rigid members, the elastic region being provided on a proximal end side of each of the pair of distal end rigid members;
an electrode configured with electrode bodies respectively protruding downward from the pair of distal end rigid members of the electrode supporting member and a spanning portion that spans between respective lower end portions of the electrode bodies; and
an operation member provided in a proximal end rigid member, and configured to move relative to the proximal end rigid member to cause the electrode to move in a direction in which the electrode protrudes relative to the pair of distal end rigid members.

2. An electrode unit configured to resect or coagulate tissue inside a subject by using a high-frequency current under observation with an endoscope, comprising:
an electrode supporting member inserted into the subject;
a proximal end rigid member joined to a proximal end of the electrode supporting member;
a distal end rigid member provided in a distal end portion of the electrode supporting member;
an elastic region having lower bending rigidity than bending rigidity of the distal end rigid member and the proximal end rigid member, the elastic region being provided in the electrode supporting member to connect between the distal end rigid member and the proximal end rigid member;
an electrode supported by the distal end rigid member and protruding from an external surface of the distal end rigid member;
an electrical connection member provided in the proximal end rigid member and electrically connected to the electrode; and
an operation member provided in the proximal end rigid member and configured to move relative to the proximal end rigid member to cause the electrode to move relative to the distal end rigid member.

3. The electrode unit according to claim 1, wherein:
the electrode is provided in an advanceable and retreatable manner in a protrusion direction of the electrode with respect to each of the pair of distal end rigid members; and
each of the pair of distal end rigid members comprises a restriction member configured to move relative to the distal end rigid member according to movement of the operation member, to change a movable range of the electrode in the protrusion direction.

4. The electrode unit according to claim 1, wherein:
a direction in which the electrode protrudes from each of the pair of distal end rigid members intersects a longitudinal axis from the proximal end to the distal end;
the operation member transmits a force causing the electrode to move in a direction along the longitudinal axis with respect to each of the pair of distal end rigid members; and
each of the pair of distal end rigid members comprises a redirection member configured to redirect the force transmitted from the operation member to the electrode, to cause the electrode to move in a direction intersecting both the protrusion direction of the electrode and the longitudinal axis.

5. The electrode unit according to claim 3, wherein:
the restriction member is connected to the operation member and is advanceable and retreatable along the longitudinal axis in a state of being in contact with a lower face of the electrode.

6. The electrode unit according to claim 4, wherein:
the redirection member is an elongated hole provided inside each of the pair of distal end rigid member and inclined upward toward the proximal end side; and
the electrode is connected to the operation member.

7. The electrode unit according to claim 1, further comprising:
the proximal end rigid member joined to a proximal end of the electrode supporting member; and
an electrical connection member provided in the proximal end rigid member and electrically connected to the electrode,
wherein:
the operation member is provided in the proximal end rigid member and is movable relative to the proximal end rigid member; and
the electrode comprises
a pair of base portions respectively supported by the pair of distal end rigid members and
a spanning portion that connects between the pair of base portions in a state of protruding to a lower side of the pair of distal end rigid members.

8. The electrode unit according to claim 6, wherein:
the pair of base portions are arranged respectively on opposed faces, being opposed to each other, of the pair of distal end rigid members.

9. The electrode unit according to claim 6, wherein:
when a pair of axes that are orthogonal to the longitudinal axis and orthogonal to each other are defined as a first axis and a second axis, with one side of the first axis being a right side and another side of the first axis being a left side, and one side of the second axis being an upper side and another side of the second axis being a lower side,
the spanning portion has a convex shape toward the lower side from the pair of base portions.

10. The electrode unit according to claim 8, wherein:
the pair of base portions protrude on the respective opposed faces toward each other along a first axis orthogonal to the longitudinal axis.

11. An endoscope system comprising:
an electrode unit configured to resect or coagulate tissue inside a subject by using a high-frequency current, including
an electrode supporting member provided with a pair of distal end rigid members, surfaces of which are covered by an electrically insulating material, and an elastic region member having lower bending rigidity than bending rigidity of each of the pair of distal end rigid members, the elastic region member being provided on a proximal end side of each of the pair of distal end rigid members,
an electrode configured with electrode bodies respectively protruding downward from the pair of distal end rigid members of the electrode supporting member and a spanning portion that spans between respective lower end portions of the electrode bodies, and
an operation member provided in a proximal end rigid member, and configured to move relative to the proximal end rigid member to cause the electrode to move in a direction in which the electrode protrudes relative to each of the pair of distal end rigid members; and
a telescope for observing an object.

12. The endoscope system according to claim 11, comprising:
a sheath into which the electrode unit is inserted;
the telescope inserted into the sheath;
a slider that holds the telescope on a proximal end side of the sheath in a movable manner in a direction along a longitudinal axis of the sheath; and
an electrode unit holding member that holds the electrode unit on the proximal end side of the sheath in a movable manner in the direction along the longitudinal axis of the sheath, independently of the telescope.
